(19) United States
(12) Patent Application Publication
(10) Pub. No.: US 2006/0231097 A1
(43) Pub. Date: Oct. 19, 2006

(54) Apparatus for CPAP therapy

(76) Inventors: Timothy R. Dougherty, Crystal, MN (US); Joseph A. Baser, Lino Lakes, MN (US); Steven J. Peterson, Eden Prairie, MN (US); Thomas A. Roman, St. Anthony Village, MN (US); Paul H. Johnson, Minneapolis, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/397,483

(22) Filed: Apr. 3, 2006

Related U.S. Application Data
(60) Provisional application No. 60/667,797, filed on Apr. 2, 2005.

(51) Int. Cl. A61M 16/00; F24F 3/14; A62B 7/00; A61M 15/00

(52) U.S. Cl. 128/204.18; 236/44 A; 128/203.12; 128/204.21

(57) ABSTRACT

The present invention provides an apparatus for the supply of air for the treatment of sleep apnea. The apparatus of the present invention includes a motor and a fan unit. The motor and fan unit are enclosed in an inner case of flexible foam plastics material. The inner case is configured to contain the motor and the fan unit, and the inner case 48 is generally biased against the motor and the fan unit. The inner case is, in turn, contained within an intermediate case made of rigid material. The intermediate case conforms to and is substantially filled by the inner case such that the inner case is generally biased against the intermediate case. Finally, the intermediate case is contained within an outer case of rigid material. The outer case surrounds the intermediate case. The outer case is generally set apart from the intermediate case so that there is a gap between the intermediate case and the outer case. The inner case, the intermediate case, and the outer case are all interconnected to form a blower housing in a manner that reduces the transmission of noise from the fan unit and from the motor to the external environment to the outer case. The apparatus may also include a humidifier in fluid communication with the volute outlet so as to impart humidity to compressed air or other gasses provided to the patient. The apparatus may include a microcontroller and may be powered by battery. The microcontroller may receive signals from a battery voltage sensor, the motor rate of rotation sensor, and a pressure sensor. The microprocessor may execute an algorithm directed to maintain a constant pressure over a range of battery voltages by regulation of the motor rate of rotation in response to the battery voltage signal and the pressure signal. The control algorithm in the microcontroller may also be arranged to adjust a duty cycle of a heater in the humidifier to adjust the level of humidification provided by the humidifier.

### CROSS-REFERENCE TO RELATED APPLICATIONS

This application claims the benefit of the filing date of U.S. Provisional Patent Application Ser. No. 60/667,797 filed Apr. 2, 2005, which is hereby incorporated herein in its entirety by reference.

### BACKGROUND OF THE INVENTION

1. Field of the Invention

The present invention relates to medical devices, and, more particularly to apparatus for providing compressed air or other gas or gas mixtures e.g. for use in continuous positive airway pressure [CPAP] therapy and the treatment of sleep apnea.

2. Description of the Related Art

It is known that applying a continuous positive airway pressure (CPAP) in the range of 4.5-10 cm water to a patient by way of the nose may prevent upper airway occlusion during sleep. CPAP apparatus has become the apparatus of choice for the treatment of chronic sleep apnea, chronic pulmonary obstruction and snoring. Many CPAP apparatus are now available.

A typical CPAP apparatus has a fan unit powered by an electric motor. The fan unit has an impeller mounted so as to rotate inside a volute. The volute has a volute inlet 84 and a volute discharge. The fan unit, the motor, and associated controls are usually encased together in a housing so as to form a blower unit. Some source of electrical power must be provided to operate the motor.

A delivery tube is used to deliver pressurized air or other gasses to the patient. The delivery tube is usually a flexible plastic tube having a proximal end and a distal end. The proximal end of the delivery tube is connected to the blower unit so as to be in fluid communication with the volute discharge. The distal end of the delivery tube is fitted to the face of a patient by a patient interface so that pressurised air produced by the fan unit is delivered via the delivery tube to the nose of the patient. Thus, the patient inhales pressurized air. The patient interface may be a mask that fits over the nose and, sometime, the mouth, nasal pieces that fit under the nose, nostril inserts into the nares, or some combination thereof. The patient interface may include features that allow the patient interface to be affixed to the patient and that maintain a proper orientation of the patient interface with respect to the patient.

Since the apparatus is to be used mainly in a bedroom or other place having a low ambient noise level to facilitate sleep, minimising the sound generated by the motor and fan unit and transmitted to the external environment to the blower unit is a long-standing design goal.

Three transmission paths for sound generated by the motor and fan unit to the environment external to the blower unit have been identified. Sound may be propagated in the direction of gas flow from the volute discharge and, hence, into the environment external to the blower unit. Sound may be propagated opposite to the direction of gas flow from the volute inlet into the environment external to the blower unit. Sound may be radiated from the housing, which may act as a panel radiator. By reducing the sound radiative properties of the housing, sound propagation into the environment external to the blower unit may be reduced.

Current apparatus may reduce the sound radiation from the housing by providing a mounting body for mounting a motor and fan unit within the housing, the mounting body being formed from a compliant material adapted to be fixed with respect to the housing. The mounting body may be fitted in compression between the housing and a cover plate. The material of the mounting body may be, for example, a polyurethane foam sufficiently open-cell to have sound absorbent properties but sufficiently rigid to provide mechanical support for the motor and fan unit. The mounting body may include a recess of complementary shape to the motor and fan for receiving and locating the motor and fan. A foam insert could be fitted into the recess so as to cover the fan unit to fit around the motor. This and other currently available apparatus radiate sound from the housing into the environment external to the housing.

Because patients may wish to utilize the CPAP apparatus in locations lacking electrical power, the ability to operate a CPAP apparatus using battery power may also be important. Currently available CPAP apparatus lack the capability of operating from battery power. An important aspect of operating a CPAP apparatus using battery power is to be able to maintain the desired pressure in the delivery tube as the battery voltage decreases.

### SUMMARY OF THE INVENTION

Apparatus and methods in accordance with the present invention may resolve one or more of the needs and shortcomings discussed above and will provide additional improvements and advantages as will be recognized by those skilled in the art upon review of the present disclosure.

The present invention provides an apparatus for the supply of air for the treatment of sleep apnea. The apparatus of the present invention includes a fan unit. The fan unit includes a volute having an inlet and an outlet with an impeller rotatably supported within the volute. A motor is connected to the impeller such that the motor may impart power to the impeller thereby causing the impeller to rotate. The motor and fan unit are enclosed in an inner case of flexible foam plastics material. The inner case is configured to contain the motor and the fan unit, and the inner case is generally biased against the motor and the fan unit. The inner case is, in turn, contained within an intermediate case of rigid material. The intermediate case conforms to and is substantially filled by the inner case such that the inner case is generally biased against the intermediate case. Finally, the intermediate case is contained within an outer case of rigid material. The outer case surrounds the intermediate case. The outer case is generally set apart from the intermediate case so that there is a gap between the intermediate case and the outer case. The inner case, the intermediate case, and the outer case are all interconnected to form a blower housing in a manner that reduces the transmission of noise from the fan unit and from the motor to the external environment to the outer case.

The apparatus according to the present invention may also include a humidifier in fluid communication with the discharge of the volute so as to impart humidity to compressed air or other gasses provided to the patient. A battery may be used as the power supply to the motor and to the humidifier. A battery voltage sensor may be included to provide a battery voltage signal corresponding to the battery voltage. A motor rotation sensor that provides a motor rate of rotation signal corresponding to the rate of rotation of the motor may also be included. The apparatus may include a pressure sensor that provides a pressure signal corresponding to the fluid pressure in a manifold or other location on the outlet side of the volute.

The apparatus may include a microcontroller. The microcontroller may receive the battery voltage signal, the motor rate of rotation signal, and the pressure signal. The microcontroller is controlled by a control algorithm directed to maintain a constant pressure signal over a range of battery voltage signals by regulation of the motor rate of rotation in response to the battery voltage signal and the pressure signal. The control algorithm in the microcontroller may also be arranged to adjust a duty cycle of a heater in the humidifier to adjust the level of humidification provided by the humidifier.

Other features and advantages of the invention will become apparent from the following detailed description, and from the claims.

### BRIEF DESCRIPTION OF THE DRAWINGS

FIG. 1A illustrates a perspective view of a portion of an apparatus according to the present invention including the two parts of the blower housing and the attachment feature that, along with the internal components, make up the blower unit;

FIG. 1B illustrates a perspective view of an apparatus according to the present invention including the blower unit, a delivery tube, and a patient interface;

FIG. 2 illustrates in an exploded view from one side aspects of an apparatus according to the present invention including the fan unit, motor, manifold, and components of the inner case and intermediate case;

FIG. 3 illustrates in an exploded view from one side aspects of an apparatus according to the present invention including the intermediate case, components of the outer case, and components that may be attached to the intermediate case;

FIG. 4 illustrates in a perspective cut-away view aspects of an apparatus according to the present invention including the impeller, volute, manifold, and motor;

FIG. 5 illustrates portions of the inner case, the intermediate case, and the outer case to demonstrate the positional relationship between the inner case, the intermediate case, and the outer case;

FIG. 6 illustrates a block diagram of a control system for the blower and humidifier; and

FIG. 7 illustrates motor control features during battery operation of the apparatus in the form of a flow diagram.

All Figures are illustrated for ease of explanation of the basic teachings of the present invention only; the extensions of the Figures with respect to number, position, relationship and dimensions of the parts to form the embodiment will be explained or will be within the skill of the art after the following description has been read and understood. Further, the exact dimensions and dimensional proportions to conform to specific force, weight, strength, flow and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with an apparatus in accordance with the present invention.

### DETAILED DESCRIPTION OF THE INVENTION

Apparatus 20 of the present invention may be used in the treatment of sleep apnea and other disorders. The Figures generally illustrate embodiments of apparatus 20 including aspects of the present inventions. The particular exemplary embodiments of the apparatus 20 illustrated in the Figures have been chosen for ease of explanation and understanding of various aspects of the present inventions. These illustrated embodiments are not meant to limit the scope of coverage but instead to assist in understanding the context of the language used in this specification and the appended claims. Accordingly, variations of apparatus 20 different from the illustrated embodiments may be encompassed by the appended claims.

The apparatus 20 of the present invention provides a fan unit 36 powered by an electric motor 56. A delivery tube 28 is connected to the fan unit 36 and to the face of the patient for delivery of pressurized air or other gasses to the patent through a patient interface 72 for the treatment of sleep apnea and other disorders.

The fan unit 36 is made up of an impeller 44 and a volute 80. The impeller 44 consists of a plurality of aerodynamic blades mounted about an impeller axis 45. The blades may be mounted about the impeller axis 45 so as to define an inner radius 46 and an outer radius 47. It is desirable to reduce noise output from the impeller 44 in the range 0-5 KHz, which is the most obtrusive and which includes low frequency mechanical vibrations that can be imparted to, for example, a table on which a portion of the apparatus 20 may rest, as well as higher frequency audible sound. The noise profile of the impeller 44 may be shifted to higher frequencies by increasing the number of blades in the impeller 44. An impeller 44 having at least 15 blades and as many as 20 blades has been found to have reduced noise output in the 0-5 KHz range.

The impeller 44 is mounted so as to rotate about the impeller axis 45 inside the volute 80. The impeller axis 45 may be vertical, horizontal, or any other convenient orientation.

The volute 80 has a volute inlet 84 for the inflow of air or other gasses and a volute outlet 88 for the discharge of compressed air or other gasses from the fan unit 36. The fan unit 36, when used for CPAP in the treatment of sleep apnea, delivers an airflow ranging from 15 to 40 litres/min at a positive pressure of up to 20 cm H₂O. The impeller 44 and volute 80 may be in a radial flow configuration, which tends to operate efficiently under these pressure and discharge conditions.

When the impeller 44 and volute 80 are in a radial flow configuration, the volute 80 is formed in its major surface with a curved region defining a bell-shaped volute inlet 84. The bell-shaped volute inlet 84 is generally proximate the impeller axis 45 so that air enters within the inner radius 46 of the impeller. The volute inlet 84 may be oriented such that air flowing into the impeller has velocity components parallel impeller axis 45. The volute outlet 88 is formed in the surface of the volute 80. In a radial flow configuration, the volute outlet 88 is positioned generally within a plate of rotation of the impeller and beyond the outer radius 47 of the impeller 44. The volute outlet 88 may be oriented to be tangential to the outer radius 47 of the impeller 44. The impeller 44 and volute 80 may also be configured so as to have a mixed flow or an axial flow character. The volute inlet 84 and the volute outlet 88 may have any convenient orientation with respect to the vertical.

The motor 56 is an electric motor 56 configured for attachment to a source of electrical power. The motor 56 may be, for example, a DC electric motor 56 drawing up to about 1 Amp at 20V and rotating at a speed of up to 20,000 rpm. The power source may be a battery or mains electricity. In addition, a unit control may be associated with the motor 56. The unit control may turn the motor 56 on and off and may regulate the speed of the motor 56. The unit control may include a microprocessor in electronic communication with various sensors, and the unit control may regulate the motor 56 in response to inputs from the various sensors as directed by a control algorithm.

The fan unit 36, motor 56, unit control may be collected together and placed inside a blower housing 40 so as to define a blower unit 24. The blower unit 24 may include features that allow for display of aspects of the unit control. The blower unit 24 may include features that allow air to pass from the external environment 71 through the blower housing 40 into the volute inlet 84. An attachment feature 22 may be provided that allows for attachment of a delivery tube 28 to the blower unit 24. The blower unit 24 may include a manifold 64 that conveys air from the volute outlet 88 to the attachment feature 22 so that an attached delivery tube 28 would be in fluid communication with the volute outlet 88, thereby allowing the transmission of pressurized air or gas from the volute outlet 88 to the delivery tube 28. A muffler may be included in the manifold 64 between the volute outlet 88 and the attachment feature 22.

The delivery tube 28 may be a flexible plastic tube having a proximal end 33 and a distal end 32. The proximal end 33 of the delivery tube 28 is connected to the blower unit 24 so as to be in fluid communication with the volute outlet 88. This could be achieved by configuring the attachment feature 22 of the blower unit 24 as a male coupling extended outward from the blower housing 40 over which a female configured proximal end 33 of the delivery tube 28 is slideably received and held in place by friction. The distal end 32 of the delivery tube 28 is connected to a patient interface 72. The patient interface 72 is fitted to the face of a patient, so that pressurised air produced by the fan unit 36 is delivered via the delivery tube 28 to the patient for inhalation by the patient.

The patient interface 72 may be a mask that fits over the patient's nose and, sometime, the mouth, nasal pieces that fit under the patient's nose, nostril inserts into the patient's nares, or some combination thereof. The patient interface 72 may include features that allow the patient interface 72 to be affixed to the patient and that maintain a proper orientation of the patient interface 72 with respect to the patient.

Because the apparatus 20 is used by a patient during sleep, the apparatus 20 must be configured so as to minimize the amount of sound produced by the fan unit 36 and by the motor 56 and to reduce the transmission of this sound to the external environment 71 to the blower housing 40. The sound radiative properties of the blower housing 40 may be reduced by making the blower housing 40 in three parts, an inner case 48, an intermediate case 52, and an outer case 68.

The inner case 48 may be composed of flexible foam plastics material. The inner case 48 is configured to contain the motor 56 and the fan unit 36. The inner case 48 surrounds the motor 56 and fan unit 36 and is generally biased against the motor 56 and the fan unit 36.

An intermediate case 52 of rigid material conforms to the inner case 48 and is placed over the inner case 48. The intermediate case 52 is substantially filled by the inner case 48 so that the inner case 48 is, to a large extent, biased against the intermediate case 52 so as to minimize any gaps between the inner case 48 and intermediate case 52. The intermediate case 52 is composed of sound-reflective plastics. The material of the intermediate case 52 may be a plastic that includes a mineral, for example, barium sulphate or talc. The mineral increases the specific gravity of the plastic to above 1.7 and typically within the range of about 1.8-2.0. Greater specific gravity values are also possible. The material of the intermediate case 52 may alternatively be filled with a metal such as copper, or the intermediate case 52 may be formed of metal such as stamped sheet metal, although the latter possibility is less preferred on the grounds of manufacturing complexity, weight and cost. The relatively high specific gravity of the material of the intermediate case 52 imparts sound reflectivity.

The outer case 68 is made of a rigid material. The outer case 68 has an interior and an exterior, the outer case 68 surrounding the intermediate case 52 and the interior of the outer case 68 set apart from the intermediate case 52 so that a gap is maintained between the intermediate case 52 and the outer case 68.

The inner case 48, the intermediate case 52, and the outer case 68 are interconnected to form a blower housing 40 in a manner that reduces the transmission of noise from the fan unit 36 and from the motor 56 to the external environment 71 to the outer case 68.

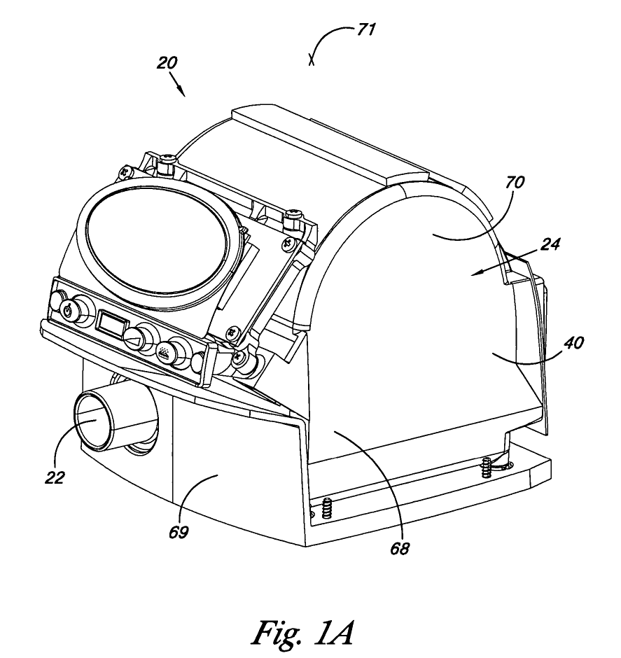

The blower housing 40 is illustrated for exemplary purposes in FIG. 1A. The blower housing 40 is illustrated as having two parts, a base 69 and a cap 70. Am attachment feature 22 is also shown. The blower housing 40, along with the internal components including the fan unit 36 and the motor 56, make up the blower unit 24. The external environment 71 outside the outer case 68 is also denoted in the Figure. FIG. 1B illustrates an apparatus 20 according to the present invention including the blower unit 24, a delivery tube 28, and a patient interface 72.

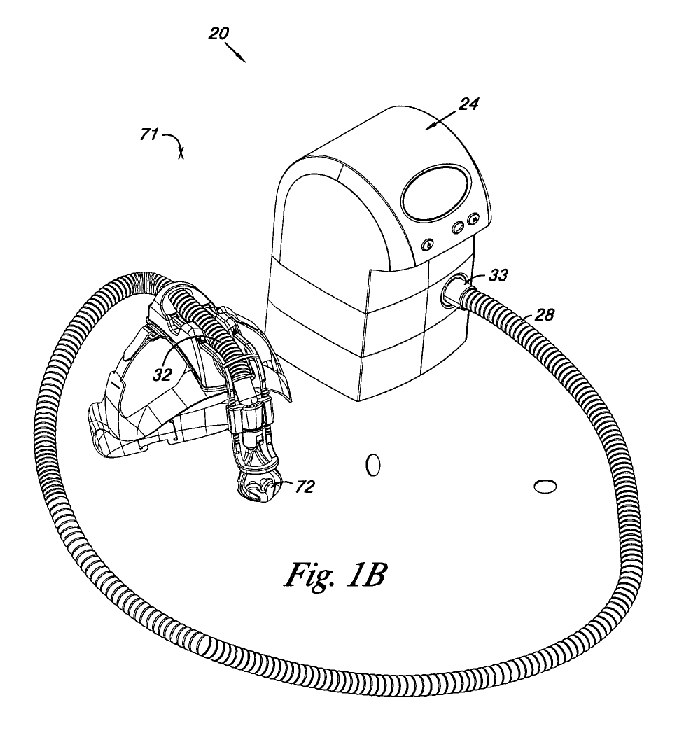

As illustrated in FIG. 2, the inner case 48 may have a motor pad 58, which may be composed of closed cell plastic foam having sufficient stiffness to support the motor 56 and fan unit 36. The motor pad 58 has a recess 57 configured to receive and bias against the motor 56. As illustrated, the fan unit 36 is located above the motor 56. The inner case 48 also has a first side-piece 49, a second side-piece 51, and a spiral top-piece 50. The first side-piece 49, the second side-piece 51, and the spiral top-piece 50 are configured so as to overlay and bias against the fan unit 36. The first side-piece 49, the second side piece 51, and the spiral top piece 50 may be formed of sound absorbant material such as an elastomeric foam with sufficient flexibility to absorb vibrations while also being sufficiently rigid to provide mechanical support to the motor 56 and fan unit 36. A 5 lb/ft³ flexible open cell polyether polyurethane foam has been found to be suitable. Air passes through the spiral top-piece 50 and into the volute inlet 84.

The first side-piece 49, as illustrated, is shaped to accommodate the volute outlet 88. The first side-piece 49, the second side-piece 51, and the spiral top-piece 50 are shown as curved and otherwise configured to conform to and be biased against the interior of the intermediate case 52. Also illustrated in FIG. 1 is a manifold 64. The manifold 64 connects to the volute outlet 88 and also to the attachment feature 22, and serves to convey compressed air between the volute outlet 88 and the attachment feature 22. A pressure tube 62 is shown connected to the manifold 64. Instruments may be mounted to the pressure tube 62 to monitor the pressure in the manifold 64.

The intermediate case 52, as illustrated in FIG. 2, is composed of a motor pad support 59 and an upper piece 53. The motor pad 58 fits within and is contained by the motor pad support 59. The upper piece 53 fits over and contains the first side-piece 49, the second side-piece 51, and the spiral top-piece 50 such that the first side-piece 49, the second side-piece 51 and the top-piece 50 are biased against the upper piece 53. The upper piece 53 locks into the motor pad support 59 by, for example, a series of bosses 66 into which stubs 67 may push fit.

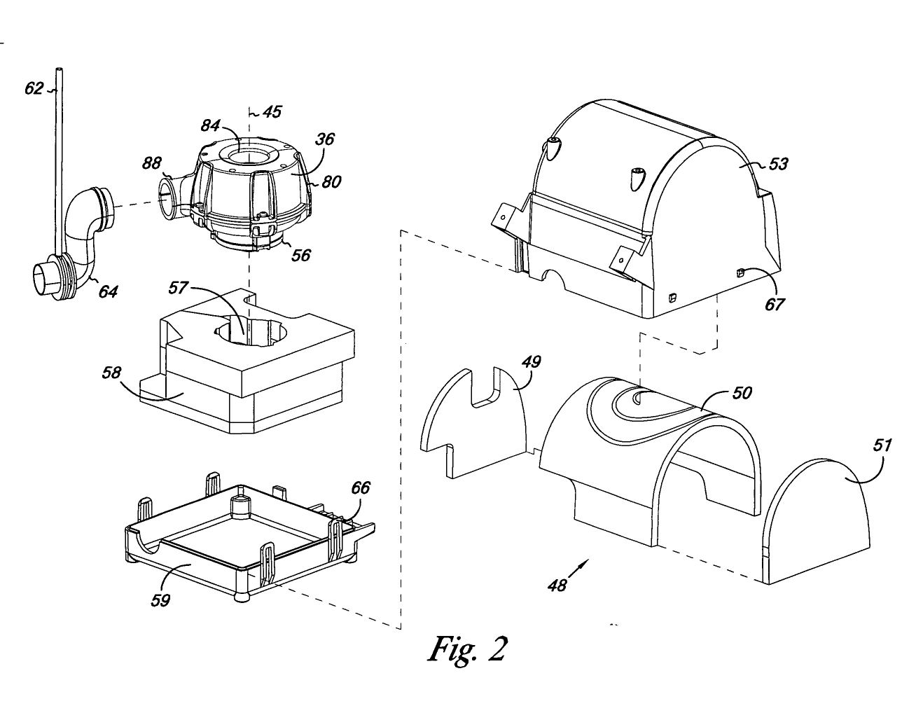

The intermediate case 52, as illustrated in FIG. 3, may also be equipped with mounting brackets 65 and similar features so that PCB's including a power PCB 77, a display PCB 78, and a switch PCB 79 may be mounted to the intermediate case 52.

As illustrated in FIG. 3, the outer case 68 may also be formed in two pieces, a base 69 and a cap 70. The cap 70 and the base 69 are configured to lock together thereby containing the intermediate case 52. The intermediate case 52 fits within the outer case 68 defined, in the illustration, by the base 69 and the cap 70. The outer case 68, as illustrated, is also configured so that the display PCB 78 and the switch PCB 79 mounted to the intermediate case 52 are visible.

The base 69 of the outer case 68, as illustrated, includes the attachment feature 22. The attachment feature 22 connects with the manifold 64. The attachment feature 22 is configured to mate with the proximal end 33 of the delivery tube 28, the delivery tube 28, the attachment feature 22, the manifold 64 may all be interconnected so as to convey compressed air from the volute outlet 88 of the fan unit 36 to the patient interface 72.

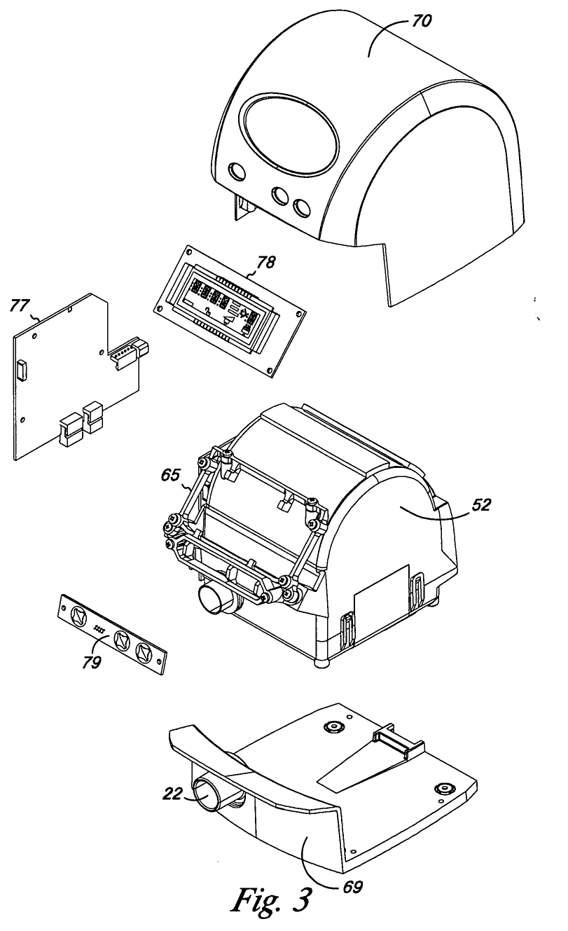

FIG. 4 illustrates a cut-away view of a fan unit 36 and motor 56. The impeller 44 is shown plus the inner radius 46 and the outer radius 47 defined by the impeller 44. The impeller axis 45 is also shown in the Figure.

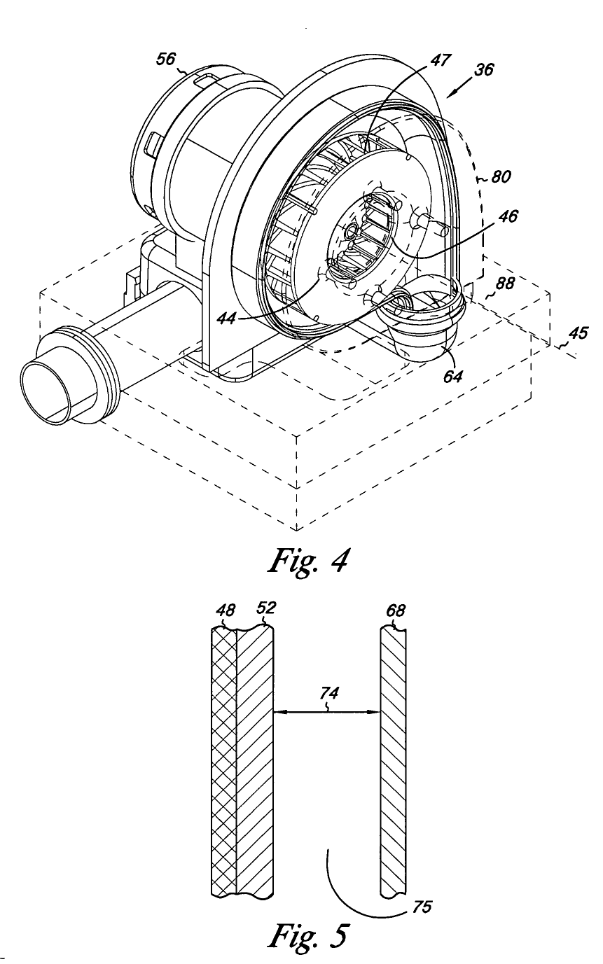

FIG. 5 illustrates portions of the inner case, the intermediate case, and the outer case to demonstrate the general relationship between the inner case 48, the intermediate case 52, and the outer case 68. As shown in FIG. 5, the inner case is biased against the intermediate case. A gap 74 is generally maintained between the intermediate case 52 and the outer case 68 so as to define an air space 75 between the intermediate case 52 and the outer case 68.

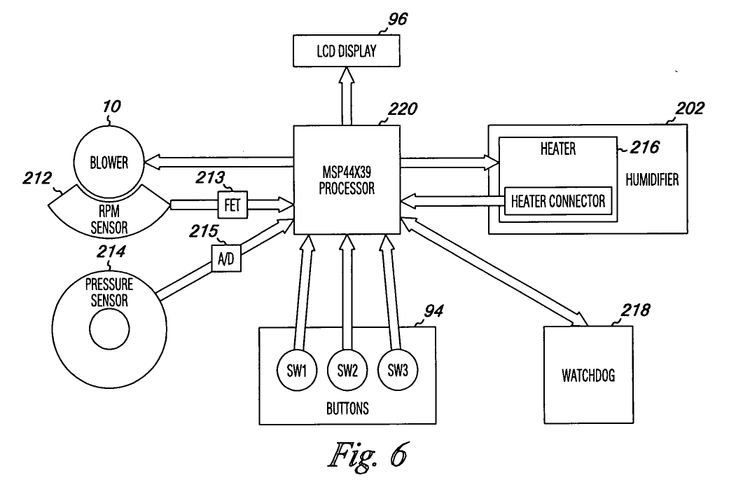

Control of the motor 56 in response to the pressure of gas in manifold 64 is illustrated in FIG. 6. An RPM sensor 212 detects a magnetic field generated by the motor 56 when the motor 56 is spinning. An FET 213 connects to the sensor 212 and the FET 213 output connects to a digital port of a microcontroller 220. The output from the FET 213 appears as a square wave. Each revolution of the motor 56 produces two positive edges. A timer internal to the microcontroller 220 measures the duration from positive edge to positive edge. A filtering/averaging routine rejects measured durations between positive edges likely to be caused by noise. A rotation rate limit routine checks if the measured duration, and hence the rotation rate, correlates to a pressure exceeding a maximum, for example, 30 cm H₂O. A pressure sensor 214 connected to pressure tube 62 is connected to an A/D converter 215, and the digitized pressure sensor output is supplied to microcontroller 220. A closed loop PID (proportional, integral, differential) algorithm uses the digitized output from pressure sensor 214 to control the rotation rate of motor 56. A PID algorithm is a form of damping control algorithm commonly used in control systems. A square wave controls the motor 56 rotation rate by pulse width modulation. A digital range of 0-255 maps 0 to a DC value at the origin, 127 to a 50% duty cycle, and 255 to a 100% duty cycle. The microcontroller 220 calculates the correct motor 56 duty cycle based on the prescribed pressure goal, the reading from the pressure sensor 214 and the coefficients of the PID algorithm and causes the appropriate square wave to be supplied to motor 56 to adjust the duty cycle to produce the required rotation rate.

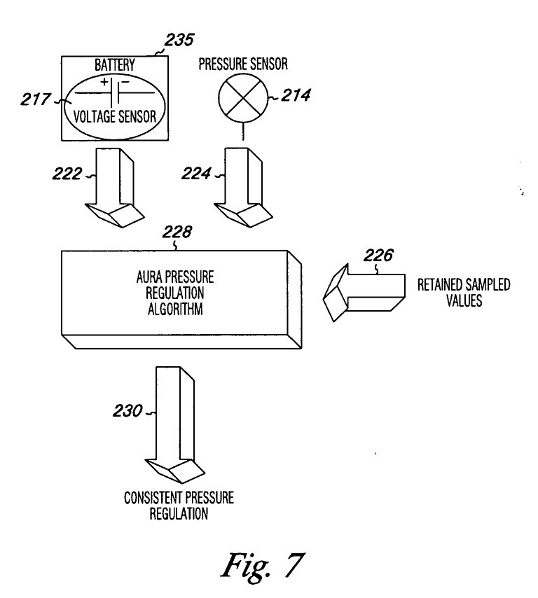

For battery operation, the microcontroller 220 executes a control algorithm 228, as illustrated in FIG. 7, having three input parameters. These three input parameters are the current pressure value 224, current battery voltage 222, and retained pressure values 226 are used to perform waveform analysis and calculate a range of future pressures using stored control algorithm 228. The control algorithm 228 monitors current battery voltage 222 and increases pulse width to provide consistent pressure regulation over a range of current battery voltages 222.

The apparatus 20 may include a humidifier 202 having a heater 216, and an algorithm in the microcontroller 220 may be arranged to adjust a duty cycle of the heater 216 by pulse width modulation to adjust the level of humidification provided by the humidifier 2-2 dependent e.g. on the pressure and/or volume of air being supplied from the fan unit 36.

The microcontroller 220 can also provide control for the heater voltage supplied to a heater 216 forming part of the humidifier 202. The control algorithm 228 may provide, for example, five levels of square wave duty cycle, which correlate to five levels of heater voltage, and, thus, correlate to five levels of humidity.

In this way, economy in power consumption can be achieved and the stored energy of a battery 235 can be used to provide power to the motor 56 and possibly power to a humidifier 202 during an overnight sleep period for a patient. The battery 235 may be fully rechargeable, the battery 235 and may have sufficient capacity to operate the system all night.

To prevent a possible shock hazard, the microcontroller 220 may connect to a circuit that measures the impedance of the heater 216. If the heater 216 does not provide proper conductance, the microcontroller 220 will immediately disable heater functionality and remove power to the heater circuitry. A watchdog circuit 218 connects to both an output port bit of the microcontroller 220 and to the reset line of the microcontroller. The watchdog circuit 218 requires the microcontroller to provide a pulse to the watchdog circuit 218 at predetermined intervals such as, for example, every 2 milliseconds. If the pulse fails to arrive within the required period, the watchdog circuit 218 resets the microcontroller 220.

The software architecture of the microcontroller 220 uses a state machine. Some states have sub-states or attributes. Examples of state are Idle (standby mode), Therapy (fan is active, algorithm regulates to pressure goal), Ramp (linear transition from standby to full pressure goal). Other states are clinician therapy setup and patient setup (view clinician prescribed settings and set humidity levels, if the device is so equipped). Sub-states exist in the setup modes for adjusting various parameters.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

1. An apparatus for the supply of air for the treatment of sleep apnea comprising:
a fan unit, wherein the fan unit includes a volute having an inlet and a discharge, and an impeller rotatably supported within the volute;
a motor connected to the impeller such that the motor may impart power to the impeller thereby causing the impeller to rotate;
an inner case of flexible foam plastics material, the inner case configured to contain the motor and the fan unit, the inner case generally biased against the motor and the fan unit;
an intermediate case of thin rigid material conforming to and substantially filled by the inner case;
an outer case of thin rigid material having an interior and an exterior, the outer case outer case surrounding the intermediate case and a gap between the intermediate case and the outer case; and,
the inner case, the intermediate case, and the outer case being interconnected to form a blower housing in a manner that reduces the transmission of noise from the fan unit and from the motor to the external environment 71 to the outer case.

2. The apparatus of claim 1, further comprising at least one PCB mounted to the intermediate case

3. The apparatus of claim 1, wherein the intermediate case is composed of a metal.

4. The apparatus of claim 1, wherein the intermediate case is composed of mineral-filled sound-reflective plastics material.

5. The apparatus of claim 4, wherein the mineral filled sound-reflective plastics material has a specific gravity of at least 1.7.

6. The apparatus of claim 4, wherein the mineral filled sound-reflective plastic material has a specific gravity from about 1.8 to about 2.0.

7. The apparatus for the supply of air for the treatment of sleep apnea comprising:
a fan unit, wherein the fan unit includes a volute having an inlet and a discharge, and an impeller rotatably supported within the volute;
a motor connected to the impeller such that the motor may impart power to the impeller thereby causing the impeller to rotate, the motor having a rate of rotation;
a blower housing, the blower housing configured to surround the fan unit and the motor;
a humidifier in fluid communication with the discharge of the volute;
a battery, the battery having a voltage and the battery supplying power to the motor;
a battery voltage sensor in electronic communication with the battery, the battery voltage sensor providing a battery voltage signal corresponding to the battery voltage;
a motor rotation sensor connected to the motor, the motor rotation sensor providing a motor rate of rotation signal corresponding to the rate of rotation;
a pressure transducer in fluid communication with the discharge of the volute, the pressure transducer providing a pressure signal corresponding to the fluid pressure;
a microcontroller, the microcontroller configured to be in electronic communication with the motor, the motor rotation sensor, the battery voltage sensor, and the pressure transducer to receive the battery voltage signal, the motor rate of rotation signal, and the pressure signal, the microcontroller controlled by a control algorithm, the control algorithm directed to maintain a constant pressure signal over a range of battery voltage signals by regulation of the motor rate of rotation in response to the battery voltage signal and the pressure signal.

8. The apparatus of claim 7, further comprising a humidifier having a heater, and wherein the control algorithm in the microcontroller is arranged to adjust a duty cycle of the heater by pulse width modulation to adjust the level of humidification provided by the humidifier.
